SEQUENCE LISTING (19 sequences)

SEQ ID NO: 1 - DNA, 6392 bp, Tomato brown rugose fruit virus-israeli
  Wild Type
gtgtattttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccagacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgcgaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcaaagctcagaacactgaggagattattaagagatggagagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggacatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccaatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggattagaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacatacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaaggcagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccaaacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggtaggggccca

SEQ ID NO: 2 - DNA, 6392 bp, Tomato brown rugose fruit virus-israeli
  60kGy-8
gtgtattttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccagacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgagaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcaaagctcagaacactgaggagattattaagagatggagagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggacatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccaatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggatttgaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacgtacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaaggcagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccaaacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggtaggggccca

SEQ ID NO: 3 - DNA, 6392 bp, Tomato brown rugose fruit virus-israeli
  60kGy-1
gtgtattttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccagacgaagttgtctgtcacagtaccttccaaacgtgtacgcaccagcaggtgaaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgagaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcaaagctcagaacactgaggagattattaagagatggagagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggacatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccaatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggatttgaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacgtacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaaggcagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccaaacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggtaggggccca

SEQ ID NO: 4 - DNA, 6392 bp, Tomato brown rugose fruit virus-israeli
  20kGy-11
gtgtattttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccagacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgatttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgagaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcaaagctcagaacactgaggagattattaagagatggagagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggacatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccaatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggatttgaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacgtacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaaggcagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccaaacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggtaggggccca

SEQ ID NO: 5 - DNA, 6392 bp, Tomato brown rugose fruit virus-israeli
  20kGy-15
gtgtattttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccaaacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgcgaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcaaagctcagaacactgaggagattattaagagatggagagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggacatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctacaccgaaggatccgatcaaaccactgattccaatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggattagaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacatacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaagacagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccaaacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactagttcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggtaggggccca

SEQ ID NO: 6 - DNA, 6392 bp, Tomato brown rugose fruit virus-israeli
  20kGy-3
gtgtattttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccagacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacgactagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgagaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcaaagctcagaacactgaggagattattaagagatggagagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggacatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccaatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggatttgaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacgtacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaaggcagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccaaacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggtaggggccca

SEQ ID NO: 7 - DNA, 6392 bp, Tomato brown rugose fruit virus-israeli
  20kGy-16
gtgtattttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccagacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgagaccaaaagatggttaattaaacctttagccaagaatcacgcatggggcgttattgaaacacatgctaggaagtaccacgttgcacttttggagtatgatgagcatggagtggtaacttgcgacggttggagaagggtggccgtgagttctgagtcaatggtttattctgatatggcaaagctcagaacactgaggagattattaagagatggagagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggacatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccaatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggatttgaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacgtacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaaggcagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccaaacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggtaggggccca

SEQ ID NO: 8 - DNA, 6392 bp, Tomato brown rugose fruit virus-israeli
  20kGy-19
gtgtattttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccagacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgagaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcaaagctcagaacactgaggagattattaagagatggagagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggacatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccaatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggatttgaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacatacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcaccctcgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaagacagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccaaacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggtaggggccca

SEQ ID NO: 9 - DNA, 6392 bp, Tomato brown rugose fruit virus-israeli
  20kGy-18
gtgtattttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccagacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgcgaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcaaagctcagaacactgaggagattattaagagatggagagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggacatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccaatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggatttgaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacgtacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaaggcagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccaaacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggtaggggccca

SEQ ID NO: 10 - DNA, 19 bp, Tomato brown rugose fruit virus-israeli
  synthetic primer F-22
ccacaacaacaaacaacaa

SEQ ID NO: 11 - DNA, 20 bp, Tomato brown rugose fruit virus-israeli
  synthetic primer R-1572
ctaatgcgtctcccgacact

SEQ ID NO: 12 - DNA, 20 bp, Tomato brown rugose fruit virus-israeli
  synthetic primer F-1070
ttacagcgcaatggaagatg

SEQ ID NO: 13 - DNA, 20 bp, Tomato brown rugose fruit virus-israeli
  synthetic primer R-2565
gcctgcttacccggtactaa

SEQ ID NO: 14 - DNA, 20 bp, Tomato brown rugose fruit virus-israeli
  synthetic primer F-2527
atggagagcctcatgtcacg

SEQ ID NO: 15 - DNA, 20 bp, Tomato brown rugose fruit virus-israeli
  synthetic primer R-4228
agctggcgtcttccttgtaa

SEQ ID NO: 16 - DNA, 20 bp, Tomato brown rugose fruit virus-israeli
  synthetic primer F-4035
ggccttgcagacgattgtgt

SEQ ID NO: 17 - DNA, 20 bp, Tomato brown rugose fruit virus-israeli
  synthetic primer R-5607
tgcaagccttacagacatcg

SEQ ID NO: 18 - DNA, 21 bp, Tomato brown rugose fruit virus-israeli
  synthetic primer F-5557
tttagtagtaaaagtgagaat

SEQ ID NO: 19 - DNA, 23 bp, Tomato brown rugose fruit virus-israeli
  synthetic primer R-CP
gatttaagtggagggaaaaacac

(19) United States
(12) Patent Application Publication
(10) Pub. No.: US 2024/0360467 A1
(43) Pub. Date: Oct. 31, 2024

(54) USE OF ATTENUATED TOMATO BROWN RUGOSE FRUIT VIRUS FOR PROTECTING CROPS AGAINST SAME VIRUS

(71) Applicant: THE STATE OF ISRAEL, MINISTRY OF AGRICULTURE & RURAL DEVELOPMENT, AGRICULTURAL RESEARCH ORGANIZATION, Rishon Lezion (IL)

(72) Inventors: Aviv DOMBROVSKY, Moshav Segula (IL); Elisheva SMITH, Tel Aviv (IL); Oded LACHMAN, Petach Tikva (IL); Neta LURIA, Ness Ziona (IL)

(21) Appl. No.: 18/289,268

(22) PCT Filed: May 4, 2022
(86) PCT No.: PCT/IL2022/050468

§ 371 Date: Nov. 2, 2023

Related U.S. Application Data
(60) Provisional application No. 63/184,827, filed on May 6, 2021.

(51) Int. Cl. C12N 15/82; C12N 7/00

(52) U.S. Cl. CPC C12N 15/8283; C12N 7/00; C12N 15/8203; C12N 2770/00063

(57) ABSTRACT

The present invention relates to the field of agriculture and more particularly to the use of attenuated ToBRFV mutants for cross-protecting Tm-2² resistant tomato varieties against ToBRFV infection and reducing disease symptoms. Also provided are methods of protecting agricultural crops such as tomatoes against said virus.

### CROSS REFERENCE TO RELATED APPLICATIONS

This application is a National Phase of PCT Patent Application No. PCT/IL2022/050468 having International filing date of May 4, 2022, which claims the benefit of priority of U.S. Provisional Patent Application No. 63/184,827, filed May 6, 2021, the contents of which are all incorporated herein by reference in their entirety.

### REFERENCE TO AN ELECTRONIC SEQUENCE LISTING

The contents of the electronic sequence listing (sequence list.txt; Size: 78,627 bytes; and Date of Creation: Dec. 5, 2023) is herein incorporated by reference in its entirety.

### BACKGROUND OF THE INVENTION

Plant viruses cause huge economic losses to the agricultural sector around the world being responsible for significant plant health issues in agriculture due to inefficient control measures available to combat these pathogens that cause damage to agricultural crops.

Tobamovirus tomato brown rugose fruit virus (ToBRFV) affects tomato, pepper and possibly other plants. ToBRFV has been first identified in Jordan and Israel in 2014 and since then, it has spread all over the world.

ToBRFV belongs to the same group as tobacco mosaic virus (TMV) and tomato mosaic virus (ToMV) albeit tomato plants tolerant to these two viruses are not tolerant to ToBRFV.

ToBRFV is a highly virulent and very aggressive virus that can cause severe infection on tomatoes with resistance genes including Tm-2², and susceptible peppers that lack the L resistance genes.

The ToBRFV virus can be easily moved from plant to plant by the farmers or by personnel coming from infected fields or greenhouses or by root-to-root contact. ToBRFV is very stable and can survive for long periods in infected fields or greenhouses, in the soil or on contaminated surfaces.

ToBRFV causes crop damages to Tm-2² resistant tomato cultivars that show disease symptoms of yellow spotted fruits with occasional severe brown rugose phenotypes. Necrotic pedicles and calyces and mosaic leaves are additional characteristic disease features. ToBRFV has all the characteristics of the stable tobamoviruses that are seed borne mechanically spread viruses, easily distributed via seeds, soil, irrigation and mechanical plant manipulation.

Leaf symptoms of ToBRFV include wrinkling and bubbling with an accompanying mosaic pattern. The fruits may be undersized with a rough surface and a browning calyx. Fruit abortion may occur while remaining fruit will be blotchy, pale and have brown, necrotic spots. Plants infected early will be stunted with poorly formed fruits while those infected later may not express fruit symptoms until the fruit turns red.

Since ToBRFV is related to TMV and ToMV, its spread and control are similar. The virus spread mechanically through people and equipment touching infected plants and transferring it to healthy plants. ToBRFV is very infectious and has high mechanical infecting ability. Tomato and other plants such as pepper are manipulated through transplanting and harvesting and are therefore prone to infection by the virus. ToBRFV is very stable, which allows it to stay infectious in the soil for a long period of time. Furthermore, there are reports of spread by bumble bee pollinators in greenhouse situations.

The worldwide spread of the potexvirus pepino mosaic virus (PepMV), newly occurring in Israel, in tomatoes has long been established infecting tomato plants via mechanical transmission. The synergism between the ToBRFV and a mild strain of PepMV amid the worldwide spread of the later poses an important challenge for finding the appropriate strategies for combating disease symptom manifestations.

Currently there are no treatments available in the form of, e.g., spray formulation that can be applied in reducing the virus's spread. Seed and transplant production are critical steps since contamination at these steps creates a risk of contaminating the plants.

Furthermore, there are no commercial tomato varieties tolerant to ToBRFV and therefore, since this virus can spread quickly and easily by mechanic transmission especially under intensive production practices, there is an urgent need for a method of protecting crops, such as tomatoes, from the ToBRFV virus.

### SUMMARY OF THE INVENTION

The present invention provides the use of attenuated ToBRFV mutants for cross-protecting Tm-2² resistant tomato varieties against ToBRFV infection and reducing disease symptoms. Also provided are methods of protecting agricultural crops such as tomatoes against said virus.

According to some embodiments of the present invention, several ToBRFV mutants have been identified that although systemically infected tomato plants were not permissive for symptom development and were effective in cross-protection studies against challenging with ToBRFV-IL or a mixture of ToBRFV-IL and mild PepMV-Israeli isolates.

According to some other embodiments of the present invention, sequencing of ToBRFV mutants are provided that show three nonsynonymous nucleotide mutations at the RNA dependent RNA polymerase coding region.

According to an embodiment of the present invention, an easy and fast strategy is provided that is immediately implemented to circumvent ToBRFV viral disease damages in crops worldwide.

According to some embodiments of the present invention, methods are provided of using attenuated ToBRFV strains for cross-protecting plants such as Tm-2² resistant tomatoes against disease manifestation of ToBRFV or a synergic mixture of ToBRFV and mild PepMV isolates.

According to some embodiments of the present invention, the tomato plants are selected from Anairis, Anna Russia, Black Krim, Black Zebra, Boludo, Cherokee Purple, Danube, Ikram, Intense, Jawara, Kumato, Montenegro, Monte Rosa, Myla, Pitenza, Retinto, Roma, San Marzano, Velasco, Vernal, Ventero, Zohara, Black Cherry, Round Cherry, Yellow Cherry and the like.

### BRIEF DESCRIPTION OF THE DRAWINGS

FIG. 1 depicts the infectious potential of ToBRFV-IL virion preparations subjected to various intensities of β-irradiation.

FIG. 2 depicts the attenuation of disease symptoms development in Tm-2² resistant tomato plants following inoculations with β-irradiated ToBRFV clones.

FIG. 3 depicts the attenuation of ToBRFV disease symptom development occurred in Tm-2² resistant tomato plants inoculated by a ToBRFV clone, harboring three non-synonymous nucleotide mutations.

FIG. 4 depicts increasing intensities of β-irradiation reduced systemic infection efficiency of the exposed ToBRFV-IL virion preparations

FIG. 5 depicts Sanger sequencing of obtained mutants

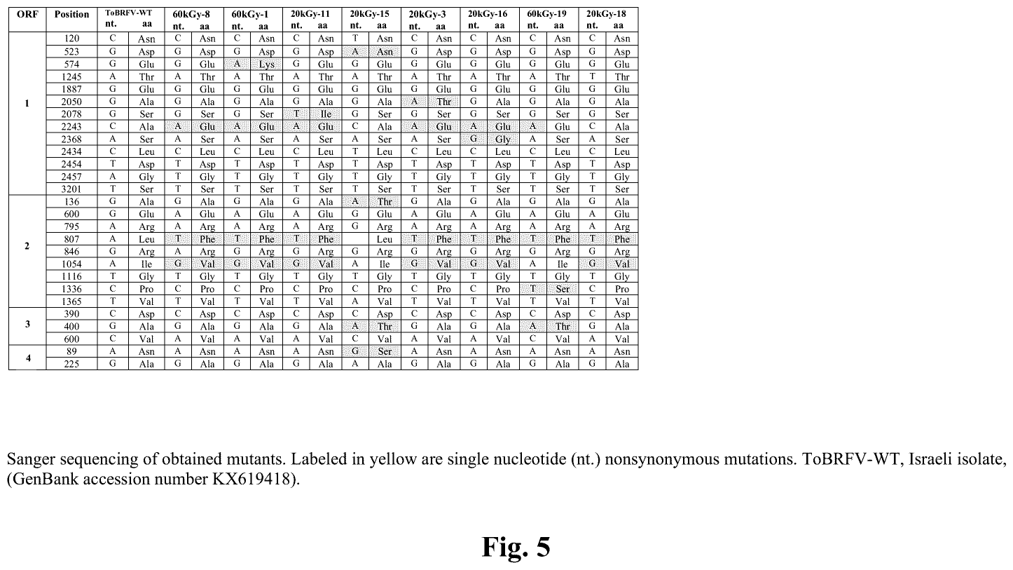

### DETAILED DESCRIPTION OF THE INVENTION

The present invention provides the use of attenuated ToBRFV mutants for cross-protecting Tm-2² resistant tomato varieties against ToBRFV infection and reducing disease symptoms. Also provided herein are methods of protecting agricultural crops such as tomatoes against said virus.

The inventors of the present invention have surprisingly uncovered that β-irradiated ToBRFV mutants can serve for cross-protection against the virus in Tm-2² resistant tomato plants. Thus, overcoming tomato disease and supplying an immediate treatment to alleviate disease symptoms may be accomplished by exposure of ToBRFV virion preparations to β-irradiation at various intensities and analyzing isolated ToBRFV clones, systemically infecting tomato plants, for disease symptom alleviation and hindrance of synergistic symptom manifestations.

According to the present invention, the term agricultural crop refers to the production of field crops such as for food use including the growth of potatoes, sweet potatoes, maize or corn, soybeans, rice, wheat, oats, barley, rye and rice, vegetables such as lettuce and cabbage, fruiting vegetables such as tomatoes, eggplant and pepper, tree fruits such as citrus, peer and plum, small fruit such as berries and cherries.

According to the present invention, the term virion refers to an entire virus particle, consisting of an outer protein shell called a capsid and an inner core of nucleic acid, wherein the core confers infectivity, and the capsid provides specificity to the virus.

According to the present invention, the term Tm-2² resistance allele refers to a resistance gene.

According to the present invention, the term rugose means wrinkled.

According to the present invention, the term dpi refers to days post inoculation.

According to the present invention, a new severe disease phenotype in Tm-2² resistant tomato cultivars has been detected in Israel, wherein the disease cause is the pathogenic effect of synergism between ToBRFV-IL and an attenuated Israel isolate of potexvirus pepino mosaic virus (PepMV), newly occurring in Israel.

According to the present invention, ToBRFV is a single-stranded positive-sense RNA virus (+ssRNA) encapsidated in a rod like shape particles ˜300 nm long. The virus has a ˜6.2 kb genome containing the four open reading frames (ORFs) encoding two subunits of RNA-dependent RNA polymerase (RdRp) complex; a short 126 kDa protein, which is associated with RNA silencing suppression and a large 180 kDa protein; a movement protein (MP) and a ˜17 kDa coat protein (CP).

According to some embodiments of the present invention, several ToBRFV mutants have been identified that although systemically infected tomato plants were not permissive for symptom development and were effective in cross-protection studies against challenging with ToBRFV-IL or a mixture of ToBRFV-IL and mild PepMV-Israeli isolates.

According to some other embodiments of the present invention, sequencing of ToBRFV mutants are provided that show three non-synonymous nucleotide mutations at the RNA dependent RNA polymerase coding region.

According to an embodiment of the present invention, an easy and fast strategy is provided that may be immediately implemented to circumvent ToBRFV viral disease damages in crops worldwide.

According to the present invention, the new disease phenotype resembles the characteristics of aggressive PepMV strains showing marbled fruits as well as the severe scarred and open unripe fruits with yellowing and narrowing leaves and shoestring-like leaves that are often observed in the mixed infected plants.

According to the present invention, the Israeli isolate of PepMV alone caused a light leaf yellowing. PepMV is a single-stranded positive-sense RNA virus (+ssRNA) with filamentous particles 508 nm long. The PepMV ˜6.4 kb genome has five ORFs encoding the RdRp complex, triple gene block proteins and a ˜26 kDa CP.

According to some embodiments, primer sets for specific identification of ToBRFV using reverse transcription are provided, wherein the obtained cDNA served for PCR amplification of the whole viral genome (˜6000 bp) using the forward primer F-22 and the reverse primer R-CP, as detailed in Table 1 below.

TABLE 1

| Name | | Primer | Amplicon |
|---|---|---|---|
| (nt. position) | Sequence (5′-3′) | set | size (bp) |
| F-22 (22) | CCACAACAACAAACAACAA | 1 | 1,550 |
| R-1572 (1,572) | CTAATGCGTCTCCCGACACT | | |
| F-1070 (1,070) | TTACAGCGCAATGGAAGATG | 2 | 1,495 |
| R-2565 (2,565) | GCCTGCTTACCCGGTACTAA | | |
| F-2527 (2,527) | ATGGAGAGCCTCATGTCAGC | 3 | 1,700 |
| R-4228 (4,228) | AGCTGGCGTCTTCCTTGTAA | | |
| F-4035 (4,035) | GGCCTTGCAGACGATTGTGT | 4 | 1,570 |
| R-5607 (5,607) | TGCAAGCCTTACAGACATCG | | |
| F-5557 (5,557) | TTTAGTAGTAAAAGTGAGAAT | 5 | 720 |
| R-CP (6,192) | GATTTAAGTGGAGGGAAAAACAC | | |

Table 2 below includes data on local lesion induction of β-irradiated purified virions of tomato brown rugose fruit virus.

TABLE 2

| β-irradiation | ^{a}Tobacco | ^{b}Necrotic local |
|---|---|---|
| intensity | plant No. | lesion No./leaf |
| 200Gray | 1 | 18, 15, 50 |
| | 2 | 60, 30, 32 |
| | 3 | 30, 11, 12 |
| 5kGray | 1 | 70, 40, 45 |
| | 2 | 30, 30, 20 |
| | 3 | 30, 35, 28 |
| 10kGray | 1 | 8, 15, 15 |
| | 2 | 3, 3, 0 |
| | 3 | 8, 6, 5 |
| 20kGray | 1 | 8, 7, 3 |
| | 2 | 2, 3, 2 |
| | 3 | 2, 1, 0 |
| 40kGray | 1 | 1, 1, 0 |
| | 2 | 0, 0, 0 |
| | 3 | 3, 1, 1 |
| 60kGray | 1 | 2, 1, 0 |
| | 2 | 3, 1, 0 |
| | 3 | 2, 1, 1 |

^{a}Nicotiana tabacum cv. Xanthi;
^{b}Data collected at 3-7 days post inoculation; Gray = β-irradiation intensity dose units.

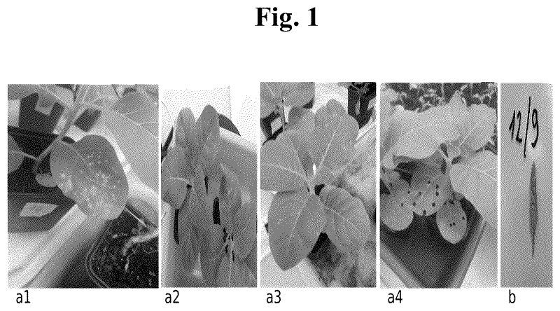

According to the present invention, as depicted in FIG. 1, the infectious potential of ToBRFV-IL virion preparations subjected to various intensities of β-irradiation is presented (G-Gray, β-irradiation intensity dose units); (a1-a4)—A gradient of necrotic local lesion (LL) development on Nicotiana tabacum cv. Xanthi directly correlated to β-irradiation intensity exposure of ToBRFV-IL; a1-10 kG, a2-20 kG, a3, a4-60 kG. a4 shows clone isolation of individual LLs.

(b) Hypersensitive response of tomato plants cv. H8009 to inoculation of several β-irradiated ToBRFV clones.

According to the present invention, the β-irradiation applied on the purified ToBRFV-IL virions differentially affects the viral genome of the viral preparation. Therefore, the necrotic LLs developed on N. tabacum cv. Xanthi represent clones of ToBRFV mutants differentially affected by the β-irradiation treatment at each of the intensities. Multiple clones of β-irradiated ToBRFV, extracted from a number of individual necrotic LLs of each intensity, were tested for systemic infection of mildly susceptible tomato plants cv. H8009.

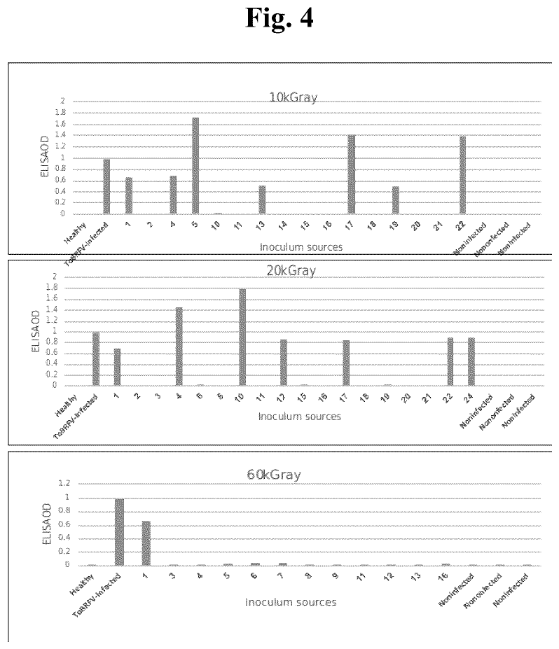

According to the present invention, as depicted in FIG. 4, increasing intensities of β-irradiation reduced systemic infection efficiency of the exposed ToBRFV-IL virion preparations. The results of β-irradiation applied on the purified ToBRFV-IL virions show that multiple clones isolated from necrotic LLs, which were induced by high intensity β-irradiated ToBRFV virions, did not show systemic infection of ToBRFV at 14 dpi, as analyzed by ELISA.

Furthermore, the inoculated leaves of the a-symptomatic plants that were negative in ELISA test showed necrosis, as depicted in FIG. 1b, and thus ToBRFV clones that did not show viral systemic infection at 14 dpi were either ToBRFV viral particles that no longer infected tomato plants or attenuated ToBRFV strains that slowly spread in the tomato plants but were infectious upon inoculation of N. tabacumcv. Xanthi.

Various isolated β-irradiated ToBRFV clones have been tested for systemic spread and symptom development in the highly susceptible tomato cv. M82 and concomitantly analyzed the efficiency of Tm-2² resistant tomato cv. Zohara in blocking/alleviating symptom development by the mutant clones.

β-irradiated ToBRFV clones, isolated after exposure of ToBRFV-IL virion preparations to increasing β-irradiation intensities, showed inversely correlated systemic infection efficiency in tomato plants cv. H8009, which were tested by ELISA, as depicted in. FIG. 4.

According to the present invention, systemic infection and symptom manifestations induced by β-irradiated ToBRFV clones were studied by inoculating the highly susceptible tomato plants cv. M82 and the Tm-2² resistant tomato plants cv. Zohara. Three groups of β-irradiated ToBRFV clones were tested, which showed three different characteristics upon inoculation of the mildly susceptible tomato plants cv. H8009:
(i) a-symptomatic plants that were ToBRFV-ELISA negative indicating lack of systemic infection and showed HR induced LLs;
(ii) a-symptomatic plants that were ToBRFV-ELISA positive indicating systemic infection;
(iii) symptomatic plants that were ToBRFV-ELISA positive indicating systemic infection.

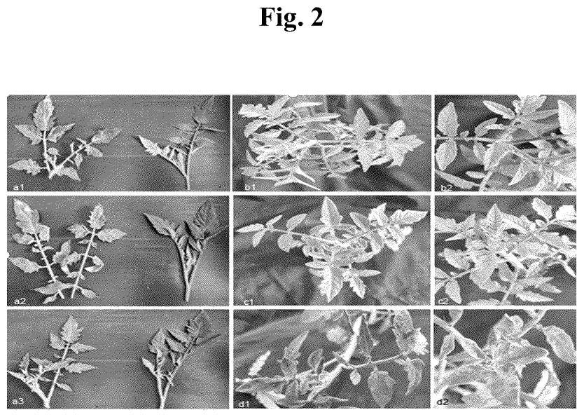

Thus, several β-irradiated ToBRFV mutants that although systemically infected Tm-2² resistant tomato cv. Zohara did not show symptoms tested at 45 dpi as depicted in FIG. 2, a1-a3 and in Table 3 below, which details the systemic spread and symptom development of β-irradiated ToBRFV clones in susceptible and Tm-2² resistant tomato plants.

TABLE 3

| | Inoculum | | | Tm-2² | |
|---|---|---|---|---|---|
| | source β- | | | resistant | |
| | irradiation | Susceptible | | Zohara | |
| Inoculum source | intensity | M-82 tomato | | tomato | |
| characteristics | identifier | plants | ELISA | plants | ELISA |
| Tomato cv. | ^{a, b}60kGray- | Severe | High | No | High |
| H8009 systemic | 13 | mosaic & | | symptoms | |
| symptoms: No | | shoestring- | | | |
| ELISA: No | | like | | | |
| HR: Yes | ^{a, b}60kGray- | Severe | High | No | High |
| | 16 | mosaic | | symptoms | |
| | 20kGray-6 | Mosaic | High | Mosaic | High |
| | | (weak) & | | (weak) | |
| | | shoestring- | | | |
| | | like (weak) | | | |
| | 20kGray-14 | Severe | High | Mosaic | High |
| | | mosaic | | (weak) | |
| | ^{b}20kGray- | Severe | High | No | High |
| | 16 | mosaic & | | symptoms | |
| | | shoestring- | | | |
| | | like | | | |
| Tomato cv. | ^{a, b}60kGray- | Severe | High | No | High |
| H8009 systemic | 8 | mosaic & | | symptoms | |
| symptoms: No | | shoestring- | | | |
| ELISA: High | | like (weak) | | | |
| | ^{b}20kGray-3 | No | High | No | Negative |
| | | symptoms | | symptoms | |
| | ^{b}20kGray- | Mosaic & | High | No | High |
| | 11 | shoestring- | | symptoms | |
| | | like (weak) | | | |
| | ^{b}20kGray- | Severe | High | No | High |
| | 15 | mosaic & | | symptoms | |
| | | shoestring- | | | |
| | | like (weak) | | | |
| | ^{b}20kGray- | Shoestring- | High | No | High |
| | 18 | like | | symptoms | |
| | 20kGray-19 | Mosaic & | High | Mosaic | High |
| | | shoestring- | | (weak) | |
| | | like | | | |
| | | (medium) | | | |
| | ^{b}10kGray-2 | Mosaic & | High | No | High |
| | | shoestring- | | symptoms | |
| | | like | | | |
| | | (medium) | | | |
| Tomato cv. | 60kGray-3 | Mosaic & | High | Mosaic | High |
| H8009 systemic | | shoestring- | | (weak) | |
| symptoms: yes | | like (weak) | | | |
| ELISA: High | | | | | |

^{a} β-irradiated ToBRFV clones analyzed for mutations;
^{b}B-irradiated ToBRFV clones subjected to challenging studies.
HR = Hypersensitive Response.

According to some embodiments of the present invention, tobamoviruses that systemically infect tomato plants, but do not cause disease symptoms, serve for cross-protection against disease causing tobamoviruses. Selected β-irradiated ToBRFV clones, which systemically infected Tm-2² resistant tomato plants cv. Zohara, have been tested by ELISA, without symptom manifestation, as detailed in Table 3.

According to the present invention, FIG. 2 depicts the attenuation of disease symptom development in Tm-2² resistant tomato plants following inoculations with β-irradiated ToBRFV clones, wherein:

(a1-3) Differential symptom manifestations in susceptible M82 (plants on the left) and Tm-2² resistant Zohara (plants on the right) tomato plants upon systemic infection with β-irradiated ToBRFV clones at 45 days post-inoculation.

The depicted inoculating β-irradiated ToBRFV clones were:
(a1), 60kGray-8;
(a2), 60kGray-13;
(a3), 10kGray-2;

(b, c and d) Differential symptom manifestations observed in Tm-2² resistant Ikram tomato plants, pre-inoculated with various β-irradiated ToBRFV clones that were tested for cross-protection efficiency towards challenging with a mixture of ToBRFV-IL and mild PepMV-Israeli isolates. The depicted challenged β-irradiated ToBRFV clones were:
(b1, b2), 60kGray-8;
(c1, c2), 20kGray-15;
(d1, d2), 10kGray-2.

According to some embodiments of the present invention, tobamoviruses that systemically infect tomato plants but do not cause disease symptoms serve for cross-protection against disease causing tobamoviruses.

According to the present invention, β-irradiated ToBRFV clones that systemically infected Tm-2² resistant tomato plants cv. Zohara have been selected and tested by ELISA, without symptom manifestation, as detailed in Table 3. The selected clones were each inoculated onto 10 Tm-2² resistant tomato plants cv. Ikram and at 10 dpi the plants were challenged with ToBRFV-IL. The challenged β-irradiated ToBRFV inoculated plants did not show symptoms up to 30 days post challenging although several β-irradiated ToBRFV inoculated plants showed systemic accumulation of ToBRFV, tested by ELISA, as detailed in Table 4. The plants were than subjected to a second challenging with a mixture of ToBRFV-IL and a mild PepMV-Israeli isolate or PepMV-Israeli isolate only, depending on the systemic accumulation of ToBRFV, in order to establish effectivity of cross-protection against the synergistic phenotype induced in mixed infected tomato plants.

According to the present invention, β-irradiated ToBRFV clones protect Tm-2² resistant tomato plants cv. Ikram against ToBRFV-IL disease symptom development, as detailed in Table 4 below.

TABLE 4

| | | Symptom | | |
|---|---|---|---|---|
| | Inoculum source | development at | ELISA at | Viral 2^{nd} |
| | β-irradiation | 30 days post | 30 days post | challenge at 30 |
| Inoculum source | intensity | ToBRFV-IL 1^{st} | ToBRFV-IL 1^{st} | days post 1^{st} |
| characteristics | identifier | challenge | challenge | challenge |
| Tomato cv. H8009 | 60kGray -13 | Negative | +7/10 | PepMV-Israeli |
| systemic | | | | isolate |
| symptoms: No | 60kGray -16 | Negative | +6/10 | *A viral mixture |
| ELISA: No | | | | |
| Tomato cv. H8009 | 60kGray -1 | Negative | Negative | *A viral mixture |
| systemic | 60kGray -8 | Negative | +9/10 | PepMV-Israeli |
| symptoms: No | | | | isolate |
| ELISA: No | 20kGray -3 | Negative | Not tested | *A viral mixture |
| | 20kGray -11 | Negative | Not tested | PepMV-Israeli |
| | | | | isolate |
| | 20kGray -15 | Negative | Negative | *A viral mixture |
| | 20kGray -18 | Negative | Negative | *A viral mixture |
| | 10kGray -2 | Negative | +2/10 | *A viral mixture |

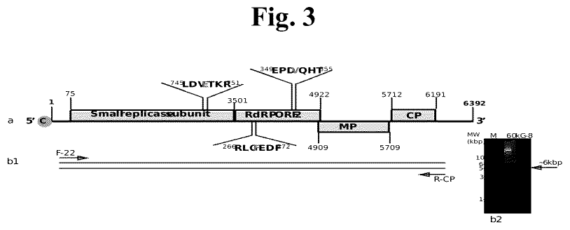

*A viral mixture contained ToBRFV-IL and a mild PepMV-Israeli isolate preparations.
Symptom development inspected at 18 days post the 2^{nd} challenging stage showed mosaic manifestations on upper leaves of the Tm-2² resistant tomato plants cv. Ikram pre-inoculated with 10kGray-2 β-irradiated ToBRFV clone whereas other challenged plants including plants pre-inoculated with 20kGray-15 and 60kGray-8 β-irradiated ToBRFV clones were a-symptomatic (FIG. 3b-d).

According to some embodiments of the present invention, sequencing of β-irradiated ToBRFV mutants systemically infected Tm-2² resistant tomato plants cvs. Zohara and Ikram that block ToBRFV disease symptom development revealed non-synonymous nucleotide substitution in the p126 small replicase subunit at the non-conserved region II, position 748 alanine (counting the first methionine).

According to the present invention, one ToBRFV mutant clone, depicted in FIG. 3, showed three non-synonymous nucleotide substitutions at the RdRp ORF1 and 2 coding regions. At the ORF1, nucleotide substitution has occurred at nt 2243: GTCTTAGATGTTGCGACCAAAAGAT to GTCTTAGATGTTGaGACCAAAAGAT, which has caused the amino acid (aa) substitution A748E. At the ORF2 two nucleotide substitutions have occurred, one at nt 807: AGACTTGGATTAGAAGATT to AGACTTGGATTIGAAGATT, which has caused the aa substitution L269F and a second substitution has occurred at nt 1054: TTTCCTGACATACAGCATACA to TTTCCTGACgTACAGCATACA, which has caused the aa substitution I352V.

Furthermore, A748E substitution, which changes a hydrophobic aa with a non-polar side chain to an acidic aa with an electrically charged side chain, has occurred at the p126 subunit that was implicated in RNA silencing suppression.

A similar substitution directed to the nonpolar alanine at position 747 of the tobamovirus TMV that was substituted with threonine with the electrically charged side chain has caused inactivation of the non-conserved site II activity as suppressor of RNA silencing.

Without wishing to be bound by any particular theory, it is assumed herein that these findings indicate that ToBRFV p126 has a crucial activity in symptom development on Tm-2² resistant tomato plants.

According to some embodiments of the present invention, methods are provided of using attenuated ToBRFV strains for cross-protecting plants such as Tm-2² resistant tomatoes against disease manifestation of ToBRFV or a synergic mixture of ToBRFV and mild PepMV isolates.

According to some embodiments of the present invention, the tomato plants are selected from Anairis, Anna Russia, Black Krim, Black Zebra, Boludo, Cherokee Purple, Danube, Ikram, Intense, Jawara, Kumato, Montenegro, Monte Rosa, Myla, Pitenza, Retinto, Roma, San Marzano, Velasco, Vernal, Ventero, Zohara, Black Cherry, Round Cherry, Yellow Cherry and the like.

### EXAMPLES

The following examples further illustrate the invention but, of course, should not be construed as in any way limiting its scope.

### Example 1

### Preparation of β-Irradiated ToBRFV Clones.

Purified ToBRFV preparation from symptomatic leaves of ToBRFV-IL inoculated tomato plants cv. Ikram, were applied on filter-paper rectangles and subjected to β-irradiation using Electron Beam (E-Beam) irradiation at six different intensities, using accelerator EBLab300. Discs (4 of 0.5 cm each), isolated from each β-irradiated viral preparation, were subjected to virus extraction in 200 μl 10 mM sodium phosphate buffer, pH=7.0. After vortex application and centrifugation, the extractions were applied on Nicotiana tabacum cv. Xanthi plants. The third fourth and fifth leaves below the apex of each plant were inoculated and inspected for necrotic local lesion (LL) development. At 10 dpi LLs were picked, each crushed in 40μl 10 mM sodium phosphate buffer, pH=7.0. Samples (20 μl) were applied onto mildly susceptible tomato plants cv. H8009, one LL extract per plant. At 14 dpi inoculated tomato plants were tested for ToBRFV by ELISA.

### Example 2

Study of Tm-2² resistance efficiency against the various β-irradiated ToBRFV clones.

β-irradiated ToBRFV clones that were applied on tomato plants cv. H8009 showed three different characteristics: (1) a-symptomatic and ELISA negative, which showed necrosis in the inoculated leaves; (2) a-symptomatic and ELISA positive; (3) symptomatic and ELISA positive. The tomato plants cv. H8009 showed a successful hypersensitive response (HR) towards several β-irradiated ToBRFV clones. The Tm-2² resistance efficiency was tested by comparing a tomato variety harboring Tm-2² to M82 variety which was susceptible to ToBRFV-IL. β-irradiated ToBRFV clones from each of the three groups were inoculated onto the highly susceptible tomato plants cv. M82 and the Tm-2² resistant tomato plants cv. Zohara. At 45 dpi plants were tested by ELISA and inspected for symptom development.

### Example 3

Cross-protection studies using β-irradiated ToBRFV clones for resistance induction against ToBRFV-IL in single infection and in a mixture of ToBRFV-IL and PepMV-Israeli isolate.

β-irradiated ToBRFV clones that systemically infected Tm-2² resistant tomato plants cv. Zohara, as tested by ELISA, but did not induce symptom development, inspected at 45 dpi, were each inoculated to 10 Tm-2² resistant tomato plants cv. Ikram. At 10 dpi the plants were challenged by inoculation with ToBRFV-IL. At 30 days post-ToBRFV-IL challenging the plants remained a-symptomatic and were tested by ELISA. Plants that were ELISA positive for ToBRFV were subjected to a 2^{nd} challenging assay by sap-mechanical inoculation with PepMV-Israeli isolate that was propagated on Datura stramonium plants. Plants challenged by ToBRFV-IL that were ELISA negative were subjected to a 2^{nd} challenging assay by inoculation with a mixture of ToBRFV-IL and PepMV-Israeli isolate preparations.

### Example 4

### Viral Purification, RT-PCR and Sequencing of β-Irradiated ToBRFV Clones.

Leaf samples of inoculated tomato plants were ground in general extraction buffer. The Accuprep Viral RNA Extraction kit was used for viral RNA extraction. For reverse transcription (RT), the qPCRBIO cDNA synthesis kit was used. The obtained cDNA served for PCR amplification of the whole viral genome (˜6000 bp) using the forward primer F-22 and the reverse primer R-CP, as detailed in Table 1. For ‘genome walking’ sequencing 5 designed primer sets were used for segment amplification and the obtained amplicons were sequenced. The authenticity of each amplified genome segment was confirmed using the Basic Local Alignment Search Tool (BLAST) search algorithm against the National Center for Biotechnology Information (NCBI) GenBank (https://blast.ncbi.nlm.nih.gov/Blast.cg) and alignment of the deduced amino acid sequence was performed using SnapGene software.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Use of β-irradiated tomato brown rugose fruit virus (ToBRFV) mutants for cross-protection against same virus in Tm-2² resistant crop plants.

2. The use of attenuated ToBRFV mutants of claim 1 for cross-protecting Tm-2² resistant tomato varieties against ToBRFV infection and for reducing disease symptoms

3. The use of attenuated ToBRFV mutants of claim 2 for cross-protecting Tm-2² resistant tomato varieties against ToBRFV infection and for reducing disease symptoms wherein said mutants are at least one single nucleotide (nt.) nonsynonymous mutations selected from Table 5

4. The use of attenuated ToBRFV mutants of claim 2 for cross-protect Tm-2² resistant tomato varieties against ToBRFV and mild PepMV mixed infections and for reducing the synergistic disease symptoms.

5. The use in claim 4 of attenuated ToBRFV mutants wherein said mutants are at least one single nucleotide (nt.) nonsynonymous mutations selected from Table 5

6. The use of attenuated ToBRFV mutants of claim 3, wherein the mutations in ToBRFV small replicase unit p126 at position 748 alanine (counting the first methionine) to other amino acid that attenuates symptom development on Tm-2² resistant tomato plants.

7. The use of attenuated ToBRFV mutants of claim 4, wherein the other amino acid is selected from alanine, arginine, asparagine, aspartic acid, cysteine, glutamine, glutamic acid, histidine, isoleucine, leucine, lysine, methionine, phenylalanine, proline, serine, threonine, tryptophan, tyrosine and valine

8. The use of attenuated ToBRFV mutants of claim 5, wherein the other amino acid is glutamic acid.

9. The use of claim 1, wherein said Tm-2² resistant crop plants are tomatoes.

10. The use of claim 7, wherein the tomato plants are selected from Anairis, Anna Russia, Black Krim, Black Zebra, Boludo, Cherokee Purple, Danube, Ikram, Intense, Jawara, Kumato, Montenegro, Monte Rosa, Myla, Pitenza, Retinto, Roma, San Marzano, Velasco, Vernal, Ventero, Zohara, Black Cherry, Round Cherry and Yellow Cherry.

11. A method of protecting plants against ToBRFV infection, which comprises using attenuated ToBRFV strains for cross-protecting the plants against disease manifestation of ToBRFV or a synergic mixture of ToBRFV and mild PepMV isolates.

12. The method of protecting plants against ToBRFV infection of claim 9, wherein the plants are Tm-2² resistant tomatoes.

13. The method of protecting plants against ToBRFV infection of claim 10, wherein the tomato plants are selected from Anairis, Anna Russia, Black Krim, Black Zebra, Boludo, Cherokee Purple, Danube, Ikram, Intense, Jawara, Kumato, Montenegro, Monte Rosa, Myla, Pitenza, Retinto, Roma, San Marzano, Velasco, Vernal, Ventero, Zohara, Black Cherry, Round Cherry and Yellow Cherry.
